(19) United States
(12) Patent Application Publication
(10) Pub. No.: US 2022/0203044 A1
(43) Pub. Date: Jun. 30, 2022

(54) STAB-PREVENTING INJECTION NEEDLE AND STAB-PREVENTING INJECTOR WITH ELASTIC PROTECTIVE CAP

(71) Applicant: ZHANJIANG JIANLIYUAN MEDICAL PRODUCTS CO., LTD., Zhanjiang, Guangdong (CN)

(72) Inventors: Rizhi CHEN, Zhanjiang, Guangdong (CN); You JIANG, Zhanjiang, Guangdong (CN); Shigeng XIE, Zhanjiang, Guangdong (CN)

(21) Appl. No.: 17/603,981

(22) PCT Filed: Nov. 9, 2020
(86) PCT No.: PCT/CN2020/127569

§ 371 Date: Oct. 15, 2021

(30) Foreign Application Priority Data
Nov. 14, 2019 (CN) 201911114489.4
Nov. 14, 2019 (CN) 201911114490.7

(51) Int. Cl. A61M 5/32; A61M 5/34

(52) U.S. Cl. CPC A61M 5/3257; A61M 2205/0216; A61M 5/3275; A61M 5/344

(57) ABSTRACT

A stab-resistant syringe needle with an elastic protective cap and a stab-resistant syringe are provided, which include the elastic protective cap. After the syringe is used to complete injection, the protective cap can be automatically ejected through simple operations to shield a steel needle, so as to achieve a purpose of preventing stab wounds.

### TECHNICAL FIELD

The present disclosure relates to the field of medical devices, and in particular, to a stab-resistant syringe needle with an elastic protective cap, and a stab-resistant syringe with a structure capable of detaching or fixing the syringe needle.

### BACKGROUND ART

Pharmaceutical injection is the most commonly used medical method, and syringes (usually equipped with syringe needles) are medical devices with the highest utilization rate. In the use process, medical staff are often stabbed by syringe needles. Although the injuries are slight skin and flesh injuries, they are very likely to cause serious consequences. For example, the medical staff may be infected with AIDS, viral hepatitis and other diseases. Therefore, medical staff expect the popularization of stab-resistant syringes.

In recent years, some patented technologies or products related to stab-resistant syringes have appeared one after another. After the injection is completed, a steel needle is pulled out or automatically ejected into a syringe barrel to achieve a stab-resistant effect. This type of syringes requires that the needle does not retract or leak during being put into the skin, and can slide and retract after the injection is completed. Therefore, they have the common features of complex structure, high manufacturing process difficulty and high cost. The Chinese patent application No. 201610675994.6 provides a product, in which a stab-resistant sheath composed of a plurality of sliding parts shields a steel needle to achieve a stab-resistant effect. Although this product overcomes some shortcomings occurring when the steel needle is accommodated into the syringe barrel to prevent stabs, the product has a more complex structure, a higher cost and is inconvenient to operate. Due to the fact that existing stab-resistant syringes are more than ten times or even dozens of times more expensive than ordinary syringes, the current utilization rate of stab-resistant syringes in China is very low, far lower than that of some developed countries in which the stab-resistant syringes have been gradually popularized.

In view of this, the development of an effectively stab-resistant and low cost syringe is of great significance to the popularization of this type of product.

### SUMMARY

In view of the shortcomings of the prior art and market requirements, the embodiments mainly aim to provide a stab-resistant syringe needle and a stab-resistant syringe, which both are equipped with an elastic protective cap. After the syringe of the present disclosure is used to complete injection, the protective cap can be automatically ejected through simple operations to shield a steel needle, so as to achieve a purpose of preventing stab wounds. The stab-resistant syringe needle and the stab-resistant syringe have simple structures and are easy to manufacture and use, and have low costs, which are extremely beneficial to the popularization of this kind of stab-resistant syringe.

Based on the same inventive concept, according to whether the steel needle can be disassembled, the stab-resistant syringes of the present disclosure may fall into two types: a stab-resistant syringe including a stab-resistant syringe needle with an elastic protective cap and a stab-resistant syringe with a fixed needle.

The above two types of stab-resistant syringes will be separately described in detail below.

### Type I

In a specific and preferred embodiment, a stab-resistant syringe needle with an elastic protective cap is provided, which can effectively play a stab-resistant role after being assembled on a syringe. Moreover, the stab-resistant syringe needles can match with most models of existing syringes for use.

Specifically, a stab-resistant syringe needle with an elastic protective cap includes a needle holder and a steel needle fixedly connected with each other, where the stab-resistant syringe needle further includes a protective cap sleeved on the needle holder;

a top end of the protective cap is closed, and a needle hole configured for the steel needle to pass through is provided at a center of an end face at the top end of the protective cap;

a spring is arranged inside the top end of the protective cap which is connected to a top end of the needle holder by the spring;

a tail end of the protective cap is an open end and is limited and clamped to a tail end of the needle holder;

the premise is that when the tail end of the protective cap is limited and clamped to the tail end of the needle holder, the spring is compressed, and the steel needle passes through the spring and then penetrates out of the needle hole; and

when the tail end of the protective cap is not limited and clamped to the tail end of the needle holder, the protective cap is automatically ejected under an elastic force of the spring to shield the steel needle in the protective cap.

In the stab-resistant syringe needle of the embodiments, the main improvement is reflected in an arrangement of the protective cap. A fixed connection between the needle holder and the steel needle may be designed and implemented with reference to the prior art. The size of the needle holder and/or the steel needle may need to be adjusted due to the introduction of the protective cap, which is also easy to determine and operate for those skilled in the art. In the embodiments, the top end of the protective cap and the top end of the needle holder each refer to an end close to a needle head of the steel needle, and the tail end refers to the other end opposite to the end.

The protective cap is sleeved on the needle holder. When the tail end of the protective cap is limited and clamped to the tail end of the needle holder, the protective cap preferably shields an entire needle holder.

The spring is arranged inside the top end of the protective cap as a core elastic member. Considering product stability and assembly convenience, the spring may be preferably arranged coaxially with the steel needle. In some embodiments, two ends of the spring may be fixedly connected to the top end of the needle holder and the top end of the protective cap respectively. It is easy to understand that the length of the spring should be enough to ensure that the steel needle is completely shielded into the protective cap in a non-limited and non-clamped state.

Considering the convenience of design and cost, the tail end of the protective cap may be limited and clamped to the tail end of the needle holder through screwing. In a preferred embodiment, an inner edge of the tail end of the protective cap may be provided with a raised internal thread, the tail end of the needle holder may be provided with a side wing which may be engaged with the internal thread, so that limitation and clamping is implemented through screwing.

In some embodiments, an outer edge of an upper side of the needle holder may be provided with a limiting wing, and a limiting movable range is formed between the side wing and the limiting wing. When the limitation and clamping are released, the internal thread section at the tail end of the protective cap slides into the limiting movable range, and the limiting wing can play a blocking role to prevent the protective cap from being separated from the needle holder and further ensure that the steel needle is shielded in the protective cap.

In some embodiments, an extended seat may be arranged at the top end of the needle holder, and one end of the spring may be fixedly sleeved on the extended seat. Without limitation, an outer edge of the extended seat may be provided with a clamping rib which is configured to connect with the spring.

In some embodiments, the stab-resistant syringe needle with an elastic protective cap according to the present disclosure further may include a protective cover, where the protective cover which may be sleeved on the top end of the protective cap. In a preferred embodiment, In the limited and clamped state, the steel needle penetrates out of the protective cap, and the sleeved protective cover can shield the exposed steel needle.

In some embodiments, an outer edge of the spring may be further provided with a shielding tube to prevent possible left and right deviation of the spring in a compressed state. An inner diameter of the shielding tube is greater than an outer diameter of a spring ring, a height of the shielding tube is preferably the same as a height of the extended seat, and a bottom end of the shielding tube is sleeved on a bottom end of the extended seat.

After the above-mentioned structure of the stab-resistant syringe needle with an elastic protective cap according to the present disclosure is learned, it is easy for those skilled in the art to determine how to manufacture the syringe needle, and the prior art can be used for flexible selection, which will not be repeated herein.

It is easy for those skilled in the art to understand that the stab-resistant syringe needle with an elastic protective cap according to the present disclosure may have two initial standby states. In one state, the tail end of the protective cap is limited and clamped to the tail end of the needle holder, the spring is compressed, and the steel needle penetrates out of the needle hole at the top end of the protective cap, but is shielded by the protective cover sleeved on the top end of the protective cap. And in the other state, the tail end of the protective cap is non-limited and non-clamped with the tail end of the needle holder, and the steel needle is shielded in the protective cap as a whole (the protective cover may be present or omitted at this time). In view of convenience of use and higher safety, the former one, i.e., the syringe needle in the limited and clamped state, is preferred.

After the structure of the stab-resistant syringe needle with an elastic protective cap according to the present disclosure is learned, it is easy for those skilled in the art to understand how to use the syringe needle with the purpose of preventing a user from being stabbed by the steel needle. The syringe needle with the initial standby state being the limited and clamped state is taken as an example. During use, the needle holder is tightened with the syringe adapter, the protective cover is removed, and injection is performed according to a routine operation of the syringe. After the injection is completed, the limitation and clamping are released, such as, through rotating the protective cap (at this time, the protective cap is automatically ejected under the elastic force of the spring and is attached to the skin at the injection site), and then the needle is pulled out. During an entire process of drawing the needle, due to the continuous elastic force of the spring, the steel needle pulled is always shielded by the protective cap until the steel needle is completely pulled out. In this way, the purpose of preventing stab wounds is achieved.

Correspondingly, the present disclosure further provides a stab-resistant syringe, including the stab-resistant syringe needle with an elastic protective cap and a syringe barrel, where the syringe needle is detachably mounted to an adapter of the syringe barrel.

### Type II

In another specific and preferred embodiment, the present disclosure provides a stab-resistant syringe with a fixed needle.

Specifically, the stab-resistant syringe with a fixed needle includes a syringe barrel with an adapter, a steel needle mounted on the adapter, and a protective cover, where the stab-resistant syringe further includes a protective cap sleeved on the adapter;

a top end of the protective cap is closed, and a needle hole configured for the steel needle to pass through is provided at a center of an end face at the top end of the protective cap;

a spring is arranged inside the top end of the protective cap which is connected to a top end of the adapter by the spring;

a tail end of the protective cap is an open end and is limited and clamped to a tail of the adapter;

the premise is that when the tail end of the protective cap is limited and clamped to the tail of the adapter, the spring is in a compressed state, the steel needle passes through the spring and then penetrates out of the needle hole in the top end of the protective cap, and the protective cover is sleeved on the top end of the protective cap to shield an exposed steel needle; and

when the tail end of the protective cap is not limited and clamped to the tail of the adapter, the protective cap ejects under an elastic force of the spring to shield the steel needle in the protective cap.

In the stab-resistant syringe with a fixed needle according to the present disclosure, the main improvement is also reflected in an arrangement of the protective cap. The syringe barrel with the adapter, the steel needle mounted on the adapter, and the protective cover may be designed and implemented with reference to the prior art. The size of the adapter and/or the steel needle may need to be adjusted due to the introduction of the protective cap, which is also easy to determine and operate for those skilled in the art. Similar to that of type I, the top end of the protective cap and the top end of the adapter each refer to an end close to a needle head of the steel needle, and the tail end refers to the other end opposite to the end.

The spring is arranged inside the top end of the protective cap as a core elastic member. Considering product stability and assembly convenience, the spring may be preferably arranged coaxially with the steel needle. In some embodiments, two ends of the spring may be fixedly connected to the top end of the adapter and the top end of the protective cap respectively. It is easy to understand that the length of the spring should be enough to ensure that the steel needle is completely shielded into the protective cap in a non-limited and non-clamped state.

Considering the convenience of design and cost, the tail end of the protective cap may be limited and clamped with the tail of the adapter through screwing. In a preferred embodiment, an inner edge of the tail end of the protective cap may be provided with a raised internal thread, the tail of the adapter may be provided with an external thread ring which may be engaged with the internal thread, such that limitation and clamping may be implemented through screwing.

In some embodiments, an outer edge of an upper side of the adapter may be provided with a limiting wing, and a limiting movable range is formed between the external thread ring and the limiting wing. When the limitation and clamping are released, the internal thread section at the tail end of the protective cap slides into the limiting movable range, and the limiting wing can play a blocking role to prevent the protective cap from being separated from the adapter and further ensure that the steel needle is shielded in the protective cap.

In some embodiments, an extended seat may be arranged at the top end of the adapter, and one end of the spring may be fixedly sleeved with the extended seat. Without limitation, an outer edge of the extended seat may be provided with a clamping rib which is configured to connect with the spring.

After the structure of the stab-resistant syringe with a fixed needle according to the present disclosure is learned, it is easy for those skilled in the art to determine how to manufacture the syringe, and the prior art can be used for flexible selection, which will not be repeated herein.

It is easy for those skilled in the art to understand that the stab-resistant syringe with a fixed needle according to the present disclosure may have two initial standby states. In one state, the tail end of the protective cap is limited and clamped to the tail of the adapter, the spring is compressed, and the steel needle penetrates out of the needle hole at the top end of the protective cap, but is shielded by the protective cover. And in the other state, the tail end of the protective cap is not limited and clamped to the tail of the adapter, and the steel needle is shielded in the protective cap as a whole (the protective cover may be present or omitted at this time). However, in view of convenience of use and higher safety, the former one, i.e., the syringe in the limited and clamped state, is preferred.

Similarly, after the structure of the stab-resistant syringe with a fixed needle according to the present disclosure is learned, it is easy for those skilled in the art to understand how to use the syringe with the purpose of preventing a user from being stabbed by the steel needle. The syringe with the initial standby state being the limited and clamped state is taken as an example. During use, the protective cover is removed, and injection is performed according to a routine operation of the syringe with a fixed needle. After the injection is completed, the limited and clamped state is removed, such as, through rotating the protective cap (at this time, the protective cap is automatically ejected under the elastic force of the spring and is attached to the skin at the injection site), and then the needle is pulled out. During an entire needle pulling process, due to the continuous elastic force of the spring, the steel needle pulled is always shielded by the protective cap until the steel needle is completely pulled out. In this way, the purpose of preventing stab wounds is achieved.

### Technical Effects of the Present Disclosure

Although the specific structures are slightly different, the overall inventive concept of the above two types is the same. Compared with the prior art, the present disclosure mainly has the following beneficial effects.

(1) Different initial standby states may be selected according to operating habits. After the injection is completed, the limited and clamped state can be easily removed, so that the protective cap is automatically ejected to shield the steel needle so as to prevent stab wounds.

(2) The products have reliable performance, overcome the shortcoming that the steel needle of the existing stab-resistant syringe is prone to sliding, have a simple structure, are easy to manufacture and use, greatly reduces costs, and are easy to promote.

(3) The stab-resistant syringe needle with an elastic protective cap can match most models of existing syringes for use and is simple and convenient to operate.

(4) The stab-resistant syringe with a fixed needle has a simple and compact structure, and is especially suitable for small dose injections such as vaccine injections and skin test needles.

### BRIEF DESCRIPTION OF THE DRAWINGS

FIG. 1 is a schematic diagram of an overall structure of a stab-resistant syringe needle with an elastic protective cap (in a limited and clamped state) according to Embodiment 1;

FIG. 2 is a schematic diagram of an overall structure of the stab-resistant syringe needle with the elastic protective cap (in a non-limited and non-clamped state) according to Embodiment 1;

FIG. 3 is a schematic diagram of an overall structure of a stab-resistant syringe needle with an elastic protective cap (in a non-limited and non-clamped state) according to Embodiment 2;

FIG. 4 is a schematic diagram of an overall structure of a stab-resistant syringe with a fixed needle (in a limited and clamped state) according to Embodiment 3;

FIG. 5 is a schematic diagram of an overall structure of the stab-resistant syringe with the fixed needle (in a non-limited and non-clamped state) according to Embodiment 3;

FIG. 6 is a schematic diagram of an overall structure of a stab-resistant syringe with a fixed needle (in a non-limited and non-clamped state) according to Embodiment 4;

FIG. 7 is a physical view of the syringe equipped with the syringe needle of the present disclosure in Application Embodiment 1 before use;

FIG. 8 is a physical view of the syringe equipped with the syringe needle of the present disclosure in Application Embodiment 1 after use;

FIG. 9 is an enlarged physical view of the syringe needle of the present disclosure in Application Embodiment 1 before and after use;

FIG. 10 is a physical view of an experimental sample used in Application Embodiment 2 before use; and

FIG. 11 is a physical view of the experimental sample used in Application Embodiment 2 after use.

### DETAILED DESCRIPTION OF THE EMBODIMENTS

The present disclosure will be further described in detail below in conjunction with the embodiments and accompanying drawings, but it should not be understood as a limitation to the protection scope of the present disclosure.

### Embodiment 1

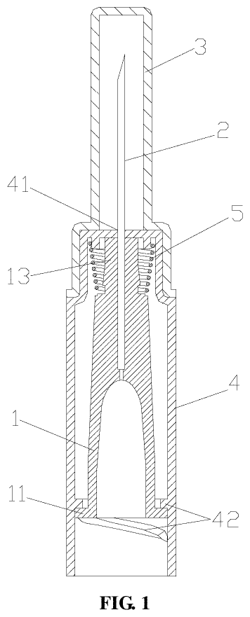

As shown in FIG. 1, a stab-resistant syringe needle with an elastic protective cap in a limited and clamped state includes a needle holder 1, a steel needle 2, a protective cover 3, and a protective cap 4.

The needle holder 1 is fixedly connected to the steel needle 2 in a conventional manner. A tail end of the needle holder 1 is provided with a raised side wing 11, a top end thereof is provided with an extended seat 13, and an outer edge of the extended seat 13 is provided with a raised clamping rib.

The protective cap 4 is sleeved outside the needle holder 1, a top end thereof is closed, and a needle hole 41 is provided at a center of an end face at the top end of the protective cap 4. And the steel needle 2 passes through the needle hole 41 and is exposed out of the protective cap 4.

The top end of the protective cap 4 is internally provided with a spring 5 (in a compressed state as shown in the figure) coaxial with the steel needle 2. One end of the spring 5 is fixedly connected to the top end of the protective cap 4, and the other end of the spring 5 is fixedly sleeved on the extended seat 13 by the clamping rib arranged on an outer edge of the extended seat 13 (the end of the spring 5 is clamped by the clamping rib).

The tail end of the protective cap 4 is an open end, and a raised internal thread 42 is provided on the inner edge of the protective cap 4. The internal thread 42 is engaged with the side wing 11. The protective cap 4 is rotated in a clockwise direction so that the internal thread 42 and the side wing 11 are positionally locked to each other, whereby the protective cap 4 and the needle holder 1 are in a state of being limited and clamped to each other.

The protective cover 3 is sleeved on the top end of the protective cap 4 to shield the exposed steel needle 2. The top end of the protective cap 4 has a circular stepped structure that is fitted to a sleeved part of the protective cover 3, and has a slot (not shown) that is fitted to the protective cover 3.

Apparently, FIG. 1 shows a typical initial standby state of the stab-resistant syringe needle with an elastic protective cap according to the present disclosure. During use, the needle holder 1 is tightened with the syringe adapter, the protective cover 3 is removed, and injection is performed according to a routine operation of the syringe. After the injection is completed, the protective cap 4 is rotated anticlockwise to release from the limited and clamped state, and then the needle is pulled out. Under the elastic force of the spring 5, the steel needle 2 pulled is always shielded by the protective cap 4 during an entire needle pulling process until the steel needle 2 is completely pulled out, so as to prevent stab wounds. In the entire process, the stab-resistant syringe needle not only has reliable performance and overcomes the shortcoming that the steel needle of the existing stab-resistant syringe is prone to sliding, but also has a simple structure, is easy to implement, and greatly reduces manufacturing and usage costs.

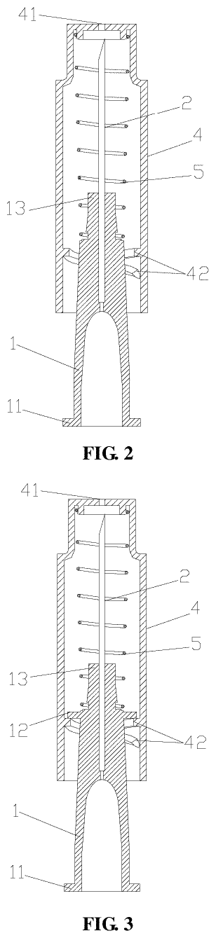

FIG. 2 shows a schematic diagram of an overall structure of the stab-resistant syringe needle with an elastic protective cap according to the present disclosure in a non-limited and non-clamped state, which is also a schematic structural diagram of the syringe needle shown in FIG. 1 after use. It can be seen that the steel needle 2 is shielded by the protective cap 4, and due to a fixed connection effect of the spring 5, the protective cap 4 will not be separated from the needle holder 1 when being ejected.

### Embodiment 2

As shown in FIG. 3, Embodiment 2 differs from Embodiment 1 only in that an outer edge of an upper side (i.e., the tail end of the extended seat 13) of the needle holder 1 is provided with a limiting wing 12, so a limiting movable range is formed between the side wing 11 and the limiting wing 12. After the limitation and clamping are released, the internal thread section at the tail end of the protective cap 4 slides into the limiting movable range. Blocked by the limiting wing 12, the internal thread section of the protective cap 4 cannot pass over the limiting wing 12, so that the protective cap 4 cannot be separated from the needle holder 1, and the protective cap 4 shields the steel needle 2 so as to prevent stab wounds.

### Embodiment 3

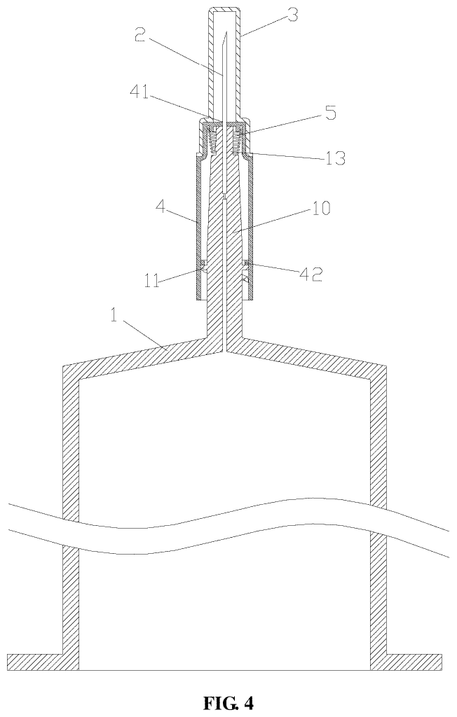

As shown in FIG. 4, a stab-resistant syringe with a fixed needle in a limited and clamped state includes a syringe barrel 1, a steel needle 2, a protective cover 3, and a protective cap 4.

The syringe barrel 1 is provided with an adapter 10. A top end of the adapter 10 is provided with an extended seat 13, and an outer edge of the extended seat 13 is provided with a raised clamping rib. And a tail is provided with a raised external thread ring 11.

The steel needle 2 is mounted on the adapter 10 of the syringe barrel 1 in a conventional manner.

The protective cap 4 is sleeved outside the adapter 10, a top end thereof is closed, and a needle hole 41 is provided at a center of an end face at the top end of the protective cap 4. The steel needle 2 passes through the needle hole 41 and is exposed out of the protective cap 4.

The top end of the protective cap 4 is internally provided with a spring 5 (in a compressed state as shown in the figure) coaxial with the steel needle 2. One end of the spring 5 is fixedly connected to the top end of the protective cap 4, and the other end thereof is fixedly sleeved with the extended seat 13 through the clamping rib on the outer edge of the extended seat 13 (the end of the spring 5 is clamped by the clamping rib).

The tail end of the protective cap 4 is an open end, and a raised internal thread 42 is provided on the inner edge of the protective cap 4. The internal thread 42 is engaged with the external thread ring 11. The protective cap 4 is rotated in a clockwise direction, so that the internal thread 42 and the external thread ring 11 are positionally locked to each other, whereby the protective cap 4 and the adapter 10 are in a state of being limited and clamped to each other.

The protective cover 3 is sleeved on the top end of the protective cap 4 to shield the exposed steel needle 2. The top end of the protective cap 4 has a circular stepped structure of a truncated cone that is fitted to a sleeved part of the protective cover 3, and has a slot (not shown) that is fitted to the protective cover 3.

Apparently, FIG. 4 shows a typical initial standby state of the stab-resistant syringe with a fixed needle according to the present disclosure. During use, the protective cover 3 is removed, and injection is performed according to a routine operation of the syringe. After the injection is completed, the protective cap 4 is rotated anticlockwise to release from the limited and clamped state, and then the needle is pulled out. Under the elastic force of the spring 5, the steel needle 2 pulled is always shielded by the protective cap 4 during an entire needle pulling process until the steel needle 2 is completely pulled out, so as to prevent stab wounds. In the entire process, the stab-resistant syringe needle not only has reliable performance and overcomes the shortcoming that the steel needle of the existing stab-resistant syringe is prone to sliding, but also has a simple structure, is easy to implement, and greatly reduces manufacturing and usage costs.

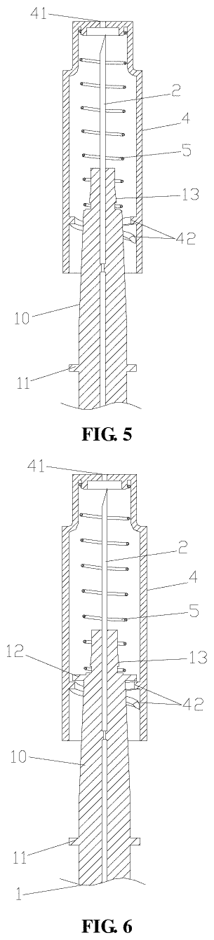

FIG. 5 shows a schematic diagram of an overall structure of the stab-resistant syringe with a fixed needle of the present disclosure in a non-limited and non-clamped state, which is also a schematic structural diagram of the syringe shown in FIG. 4 after use. It can be seen that the steel needle 2 is shielded by the protective cap 4, and due to the fixed connection effect of the spring 5, the protective cap 4 will not be separated from the adapter 10 when being ejected.

### Embodiment 4

As shown in FIG. 6, Embodiment 4 differs from Embodiment 3 only in that an outer edge of an upper side (i.e., the tail end of the extended seat 13) of the adapter 10 is provided with a limiting wing 12, and a limiting movable range is formed between the external thread ring 11 and the limiting wing 12. After the limitation and clamping are released, an internal thread section at the tail end of the protective cap 4 slides into the limiting movable range. Blocked by the limiting wing 12, the internal thread section of the protective cap 4 cannot pass over the limiting wing 12, so that the protective cap 4 cannot be separated from the adapter 10, and the protective cap 4 shields the steel needle 2 so as to prevent stab wounds.

After the structure of the product of the present disclosure is understood, the technical effects and performance advantages of the embodiments can be fully understood based on the structure. To further verify the effects of the present disclosure, the applicant performed application experiments with actual products.

### Application Embodiment 1

1. Experimental Place

The experiment was performed in Biomedical Laboratory of ZHANJIANG JIANLLYUAN MEDICAL PRODUCTS Co., Ltd.

2. Experimental Sample

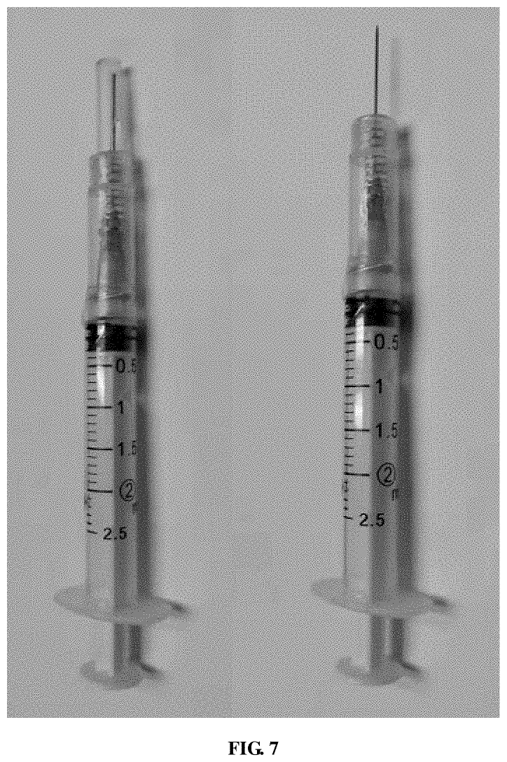

A syringe equipped with a stab-resistant syringe needle with an elastic protective cap shown in FIG. 7 was used as an experimental sample, and a specification of the syringe is 2.5 mL. In the figure, the figure on the left is a physical view of a sample with a protective cover, and the figure on the right is a physical view of the sample with the protective cover removed.

3. Experimental Subject

The subjects to be tested were rabbits for medical experiments.

4. Experimental Method

In step (1), the protective cover of the syringe was removed and experimental pharmaceutical liquid was sucked into the syringe.

In step (2), a steel needle was put into the tested subject for a routine injection operation.

In step (3), after the injection was completed, the protective cap was rotated anticlockwise until the limited and clamped state of the protective cap and a needle holder was released (the protective cap was rotated by about one-third of a turn). At this time, the protective cap is automatically ejected under an action of a spring, to contact with the skin of the subject at an injection site, and then the needle was pulled out. The steel needle pulled was always shielded by the protective cap during an entire needle pulling process until the steel needle was completely pulled out.

5. Experimental Results

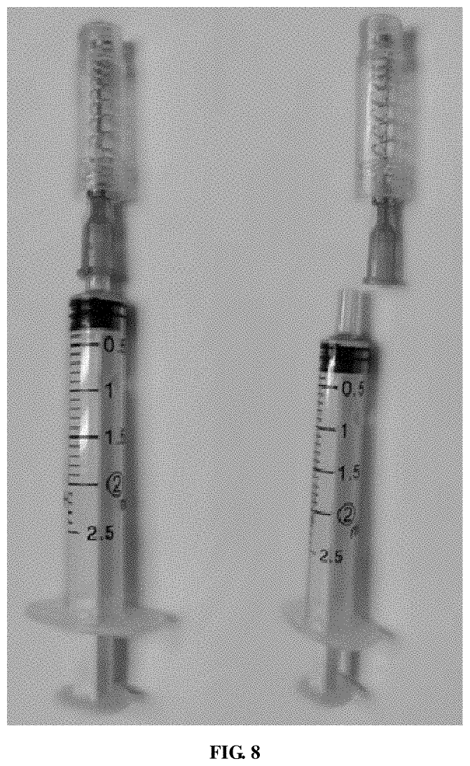

As shown in FIG. 8, the figure on the left is a physical view of an experimental sample after use, and the figure on the right is a physical view of the stab-resistant syringe needle with the elastic protective cap and a syringe adapter that are in a separated state after use.

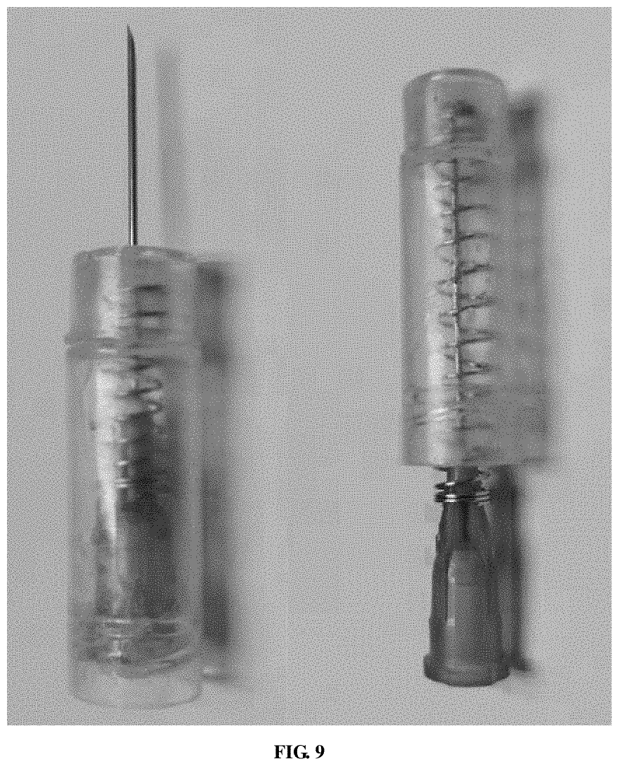

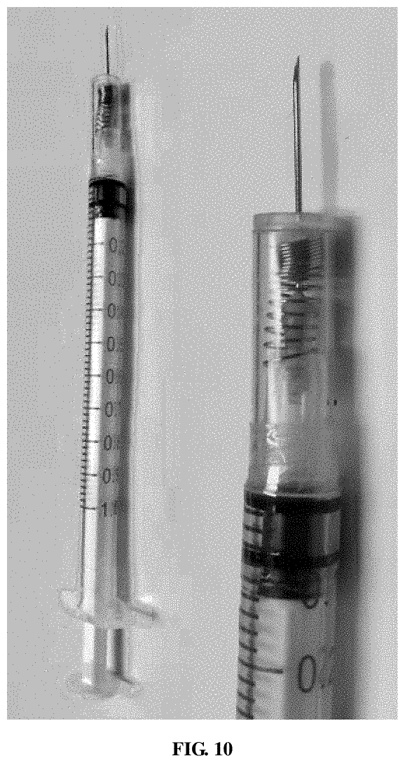

Further, FIG. 9 illustrates a physical view of the stab-resistant syringe needle with the elastic protective cap of the present disclosure (excluding a protective cover) before and after use.

In a process of using the stab-resistant syringe needle with the elastic protective cap of the present disclosure, except the step in which a pharmaceutical liquid is sucked through a needle head, the steel needle is always in a state of being shielded in the subsequent injection and needle pulling step, and the waste placement and disposal step after use. There is no hidden danger of stab wounds even if a movement range for the action is relatively large, thereby preventing stab wounds.

### Application Embodiment 2

1. Experimental Place

The experiment was performed in Biomedical Laboratory of ZHANJIANG JIANLLYUAN MEDICAL PRODUCTS Co., Ltd.

2. Experimental Sample

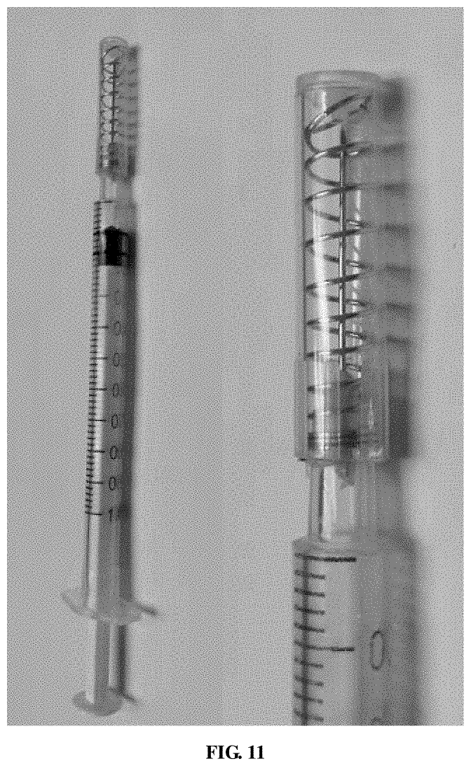

A stab-resistant syringe with a fixed needle shown in FIG. 10 was used as an experimental sample, and a specification of the stab-resistant syringe is 1 mL (for the purpose of photographing the steel needle clearly, the protective cover was removed in advance). In the figure, the figure on the left is an overall physical view, and the figure on the right is a partially enlarged physical view.

3. Experimental Subject

The subjects to be tested were rabbits for medical experiments.

4. Experimental Method.

In step (1), the protective cover of the stab-resistant syringe with a fixed needle was removed and experimental pharmaceutical liquid was sucked into the syringe.

In step (2), a steel needle of the stab-resistant syringe with a fixed needle was put into a tested subject for a routine injection operation.

In step (3), after the injection was completed, the protective cap was rotated anticlockwise until a limited and clamped state of the protective cap and an adapter was released (the protective cap was rotated by about one-third of a turn). At this time, the protective cap is automatically ejected under an action of a spring, to contact with the skin of the subject at an injection site, and then the needle was pulled out. The steel needle pulled was always shielded by the protective cap during an entire needle pulling process until the steel needle was completely pulled out.

5. Experimental Results

As shown in FIG. 11, the figure on the left is a physical view of a stab-resistant syringe with a fixed needle after use, and the figure on the right is a partially enlarged physical view.

Through the use of the stab-resistant syringe with the fixed needle of the present disclosure, except the step in which a pharmaceutical liquid is sucked through a needle head, the steel needle is always in a state of being shielded, in the subsequent injection and needle withdrawal step, and the waste placement and disposal step after use. There is no hidden danger of stab wounds even if the movement range of the action is relatively large, thereby preventing stab wounds.

The above are only exemplary embodiments of the present disclosure. Through the above content, those skilled in the art can make changes and modifications without departing from the technical concept of the present disclosure. The protection scope of the present disclosure is not limited to the above-mentioned embodiments, but is subject to the scope defined by the claims.

1-18. (canceled)

19. A stab-resistant syringe needle with an elastic protective cap, comprising a needle holder and a steel needle fixedly connected with each other, and a protective cover, wherein the stab-resistant syringe needle further comprises a protective cap sleeved on the needle holder;
an extended seat is arranged at a top end of the needle holder, and an outer edge of the extended seat is provided with a clamping rib;
a top end of the protective cap is closed, and a needle hole configured for the steel needle to pass through is provided at a center of an end face at the top end of the protective cap;
a spring is arranged inside the top end of the protective cap and is arranged coaxially with the steel needle, an end of the spring is fixedly connected to the top end of the protective cap, and another end of the spring is fixedly sleeved on the extended seat by the clamping rib provided at the outer edge of the extended seat;
a tail end of the protective cap is an open end, an inner edge of the tail end of the protective cap is provided with a raised internal thread, the tail end of the needle holder is provided with a side wing which is engaged with the raised internal thread such that limitation and clamping is implemented through screwing;
an outer edge of an upper side of the needle holder is provided with a limiting wing, and a limiting movable range is formed between the side wing and the limiting wing;
when the tail end of the protective cap is limited and clamped to the tail end of the needle holder, the spring is compressed, the steel needle passes through the spring and then penetrates out of the needle hole, and the protective cover is sleeved on the top end of the protective cap to shield an exposed steel needle;
when the tail end of the protective cap is not limited and clamped to the tail end of the needle holder, the protective cap is automatically ejected under an elastic force of the spring to shield the steel needle in the protective cap; and
during use, the needle holder is tightened with a syringe adapter, the protective cover is removed, and injection is performed according to a routine operation of the syringe; after the injection is completed, a limitation and clamping are released through rotating the protective cap; the protective cap is automatically ejected under an elastic force of the spring and is attached to skin at an injection site, and the steel needle is pulled out; during an entire process of drawing the needle, due to a continuous elastic force of the spring, the steel needle pulled is always shielded by the protective cap until the steel needle is completely pulled out.

20. The stab-resistant syringe needle with the elastic protective cap according to claim 19, wherein an outer edge of the spring is provided with a shielding tube.

21. A stab-resistant syringe with a fixed needle, comprising a syringe barrel with an adapter, a steel needle mounted on the adapter, and a protective cover, wherein the stab-resistant syringe further comprises a protective cap sleeved on the adapter;
a top end of the protective cap is closed, and a needle hole configured for the steel needle to pass through is provided at a center of an end face at the top end of the protective cap;
a spring is arranged inside the top end of the protective cap and is arranged coaxially with the steel needle, and two ends of the spring are fixedly connected to a top end of the adapter and the top end of the protective cap respectively;
a tail end of the protective cap is an open end and is limited and clamped to a tail of the adapter through screwing;
when the tail end of the protective cap is limited and clamped to the tail of the adapter, the spring is in a compressed state, the steel needle passes through the spring and then penetrates out of the needle hole in the top end of the protective cap, and the protective cover is sleeved on the top end of the protective cap to shield an exposed steel needle;
when the tail end of the protective cap is not limited and clamped to the tail of the adapter, the protective cap is ejected under an elastic force of the spring to shield the steel needle in the protective cap; and
during use, the protective cover is removed, and injection is performed according to a routine operation of the syringe with a fixed needle; after the injection is completed, a limited and clamped state is removed through rotating the protective cap, the protective cap is automatically ejected under an elastic force of the spring and is attached to skin at an injection site, and the steel needle is pulled out; during an entire needle pulling process, due to a continuous elastic force of the spring, the steel needle pulled is always shielded by the protective cap until the steel needle is completely pulled out.

22. The stab-resistant syringe with the fixed needle according to claim 21, wherein an inner edge of the tail end of the protective cap is provided with a raised internal thread, the tail of the adapter is provided with an external thread ring which is engaged with the raised internal thread, such that limitation and clamping is implemented through screwing.

23. The stab-resistant syringe with the fixed needle according to claim 22, wherein an outer edge of an upper side of the adapter is provided with a limiting wing, and a limiting movable range is formed between the external thread ring and the limiting wing.

24. The stab-resistant syringe with the fixed needle according to claim 21, wherein an extended seat is arranged at the top end of the adapter, and one end of the spring is fixedly sleeved on the extended seat.

25. The stab-resistant syringe with the fixed needle according to claim 24, wherein an outer edge of the extended seat is provided with a clamping rib which is configured to connect with the spring.

26. A stab-resistant syringe with a detachable needle, comprising the stab-resistant syringe needle with the elastic protective cap according to claim 19, wherein the stab-resistant syringe further comprises a syringe barrel, and the syringe needle is detachably mounted to an adapter of the syringe barrel.
